(19) United States
(12) Patent Application Publication
(10) Pub. No.: US 2013/0261070 A1
(43) Pub. Date: Oct. 3, 2013

(54) ANTI-EPILEPTOGENIC AGENTS

(71) Applicants: John L. Magnani, Gaithersburg, MD (US); Rachel K. King, Cabin John, MD (US)

(72) Inventors: John L. Magnani, Gaithersburg, MD (US); Rachel K. King, Cabin John, MD (US)

(21) Appl. No.: 13/877,633

(22) PCT Filed: Oct. 4, 2011
(86) PCT No.: PCT/US11/54804

§ 371 Date: Jun. 17, 2013

Related U.S. Application Data
(60) Provisional application No. 61/389,572, filed on Oct. 4, 2010.

(51) Int. Cl. C07H 15/26

(52) U.S. Cl. CPC C07H 15/26
USPC 514/35

(57) ABSTRACT

Methods and medicaments therefor are provided for altering an epileptic syndrome in an individual. Methods and medicaments therefor are also provided for preventing an epileptic syndrome in an individual at risk for developing an epileptic syndrome. More specifically, the use of particular glycomimetics for the treatment or prevention is described.

### CROSS-REFERENCE TO RELATED APPLICATION

This application claims the benefit under 35 U.S.C. §119(e) of U.S. Provisional Patent Application No. 61/389,572 filed Oct. 4, 2010; where this provisional application is incorporated herein by reference in its entirety.

### BACKGROUND

1. Technical Field

The present invention relates generally to methods for altering an epileptic syndrome or preventing an epileptic syndrome, and more specifically to the use of particular glycomimetics for the treatment or prevention.

2. Description of the Related Art

Epilepsy is one of the most common neurological problems, with up to about 1% of the population afflicted. Epileptogenesis is a term used to refer to the process of a normal brain becoming epileptic in the first place. In the process (which may occur after acute brain injury), lesions and changes in the brain progress to the formation of chronic seizures. Acute injury to the brain can arise, for example, from traumatic physical brain injury (i.e., closed head injury), stroke or infection. The term epileptogenesis is also used for the process of how a mildly epileptic brain can worsen. While the reduction or prevention of seizures has understandably been the focus of substantial medical research, one ultimately would like to prevent epilepsy or stop its progression by the development of an anti-epileptogenic agent.

The term “epilepsy” as commonly used includes more than one type of disorder, and in its generic meaning is better termed “epileptic syndromes.” An example of an epileptic syndrome is Rasmussen's syndrome.

Rasmussen's syndrome was first described in 1958 and remains an unresolved medical problem. This devastating disorder afflicts mainly children and can destroy a cerebral hemisphere. Progressive neurological deterioration (including brain atrophy) and seizures are associated with Rasmussen's syndrome. Medical treatment has typically included anticonvulsant therapy and hemispherectomy surgery where half of the brain is removed. The surgery has been more effective than anti-seizure drugs in stopping the seizures. However, side effects of the surgery typically include the addition of a limp to walking and running, and on the side opposite to the surgery there is significant impairment of hand function and loss of fine motor skills.

Accordingly, there is a need in the art for improved treatment of Rasmussen's syndrome, or prevention of Rasmussen's syndrome or its progression. The present invention fulfills these needs and further provides other related advantages.

### BRIEF SUMMARY

Briefly stated, methods for altering an epileptic syndrome, and methods for preventing an epileptic syndrome, are provided. In the present invention, the compounds used for treatment and for prevention comprise, or consist of, a particular glycomimetic. Such a compound may be combined with a pharmaceutically acceptable carrier or diluent to form a pharmaceutical composition.

In one embodiment, the present invention provides a method for altering an epileptic syndrome by treatment of an individual who is in need thereof, comprising administering to the individual a compound in an amount effective for treatment, the compound with the formula:

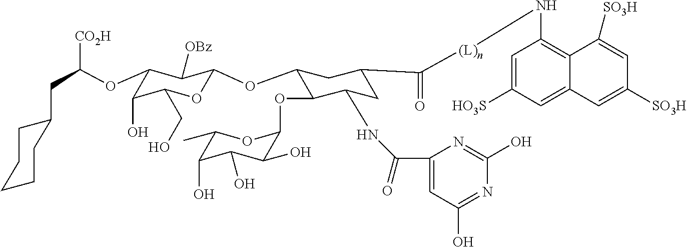

wherein

L=linker group; and

n=0-1.

In an embodiment, the present invention provides a method for preventing an epileptic syndrome in an individual at risk for developing an epileptic syndrome, comprising administering to the individual a compound in an amount effective for the prevention, the compound with the formula:

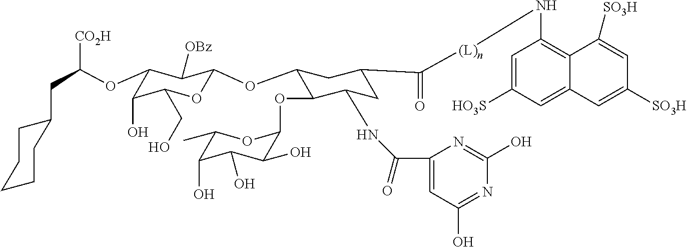

wherein

L=linker group; and

n=0-1.

In an embodiment, the above compounds are in combination with a pharmaceutically acceptable carrier or diluent.

In an embodiment, the epileptic syndrome is Rasmussen's syndrome.

In other embodiments, the above compounds or compositions thereof may be used in the manufacture of a medicament, for any of the uses recited herein.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 is a diagram illustrating the synthesis of a component of Compound #1.

FIG. 2A-2C is a diagram illustrating the synthesis of a component of Compound #1.

FIG. 3 is a diagram illustrating the modification of the component of FIG. 1.

FIG. 4 is a diagram illustrating the reaction of the components of FIGS. 2 and 3 to form Compound #1 (also referred to hereinafter as “Cmpd. #1”). Compound XIX of FIG. 2 is reacted with ethylenediamine (EDA) to form EDA-XIX.

FIG. 5 is a diagram illustrating the synthesis of Compound #2 (also referred to hereinafter as “Cmpd. #2”). Compound XIX of FIG. 2 is reacted with ethylenediamine (EDA) to form EDA-XIX.

FIG. 6 contains images from intravital microscopy of migration of neutrophils in the central nervous system (CNS) after induction of status epilepticus, and bar graphs showing rolling cells and arrested cells with and without exposure of the cells to Cmpd. #2.

FIG. 7 depicts bar graphs showing the percent inhibition by Cmpd. #2 of the rolling and adhesion (“Arrest”) of T_{H}1 cells on the blood brain microvascular after induction of status epilepticus.

FIG. 8 (FIG. 8A, FIG. 8B and FIG. 8C) shows the effects of intermittent dosing of Cmpd. #2 on the treatment of mice after inducing status epilepticus. FIG. 8A depicts bar graphs showing the mean number of seizures in mice exposed or not exposed to Cmpd. #2. FIG. 8B depicts bar graphs showing the percentage of mice with chronic seizures in mice exposed or not exposed to Cmpd. #2. FIG. 8C depicts bar graphs showing the mean duration of seizures in mice exposed or not exposed to Cmpd. #2.

### DETAILED DESCRIPTION

As noted above, the present invention provides methods for altering an epileptic syndrome, and methods for preventing an epileptic syndrome.

Compounds useful in the compositions (including medicaments) and methods of the present invention include embodiments with the formula:

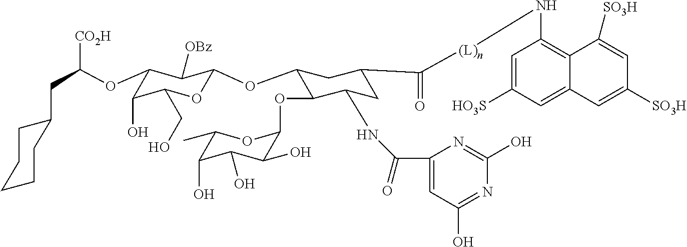

In the above formula, “L” represents a linker. There may be no linkers present (i.e., “n” is 0) or a linker may be present (i.e., “n” is 1). Where no linker is present, the compound is with the formula:

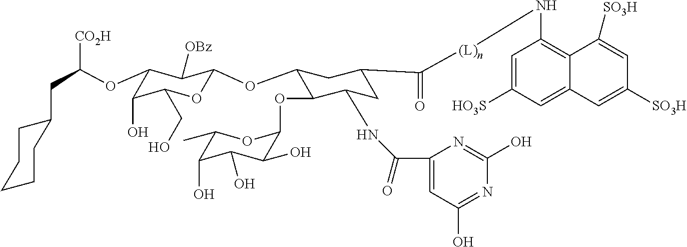

Where n is 1, a linker is present. A linker may be (or may include) a spacer group, such as —(CH₂)ₚ— or —O(CH₂)ₚ— where p is generally about 1-20 (including any whole integer range therein). Other examples of spacer groups include a carbonyl or carbonyl containing group such as an amide. An embodiment of such spacer groups is

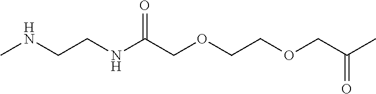 [SMILES: CNCCNC(=O)COCCOCC(C)=O]

which produces:

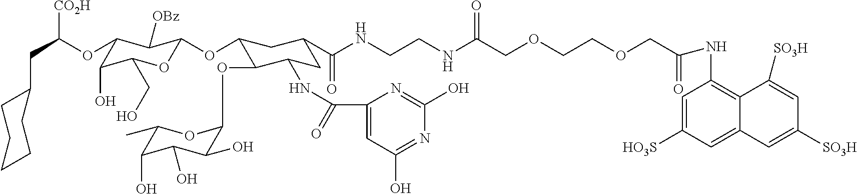

Embodiments of linkers include the following:

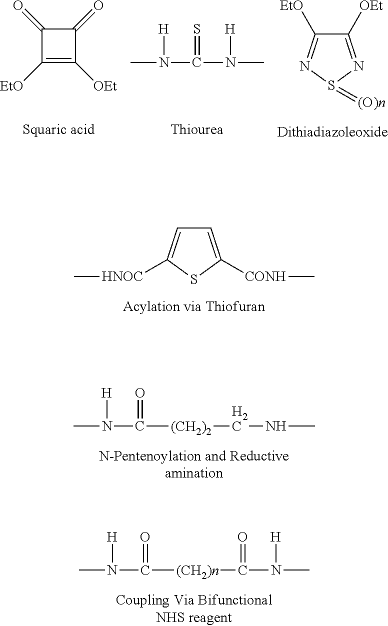

Other linkers, e.g., polyethylene glycols (PEG) or —C(═O)—NH—(CH₂)ₚ—C(═O)—NH₂ where p is as defined above, will be familiar to those in the art or in possession of the present disclosure.

In another embodiment, the linker is

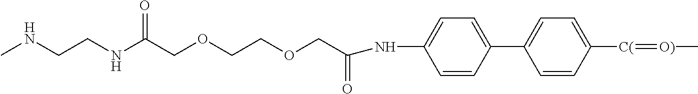

which produces:

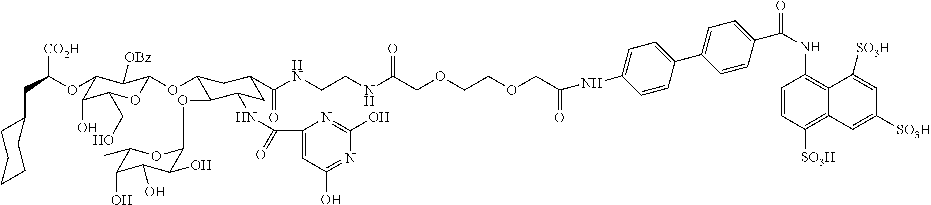

In another embodiment, the linker is

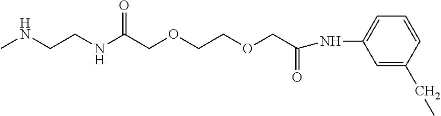 [SMILES: CCc1cccc(NC(=O)COCCOCC(=O)NCCNC)c1]

which produces:

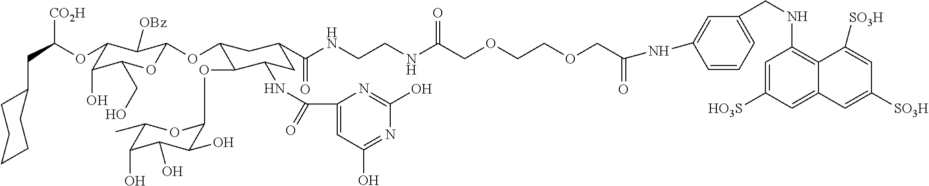

All compounds of the present invention or useful thereto (e.g., for pharmaceutical compositions or methods of treating), include physiologically acceptable salts thereof. Examples of such salts are Na, K, Li, Mg, Ca and Cl. Chemical abbreviations used herein have their normal meaning in the art, e.g., “Bz” is benzoyl which is C₆H₅C(═O)—.

Compounds as described herein may be present within a pharmaceutical composition. A pharmaceutical composition comprises one or more compounds in combination with (i.e., not covalently bonded to) one or more pharmaceutically or physiologically acceptable carriers, diluents or excipients. Such compositions may comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Within yet other embodiments, compositions of the present invention may be formulated as a lyophilizate. Compositions of the present invention may be formulated for any appropriate manner of administration, including for example, topical, oral, nasal, intravenous, intracranial, intraperitoneal, subcutaneous, or intramuscular administration.

The compositions described herein may be administered as part of a sustained release formulation (i.e., a formulation such as a capsule or sponge that effects a slow release of compound following administration). Such formulations may generally be prepared using well known technology and administered by, for example, oral, rectal or subcutaneous implantation, or by implantation at the desired target site. Carriers for use within such formulations are biocompatible, and may also be biodegradable; preferably the formulation provides a relatively constant level of compound release. The amount of compound contained within a sustained release formulation depends upon the site of implantation, the rate and expected duration of release and the nature of the condition to be treated or prevented.

The above described compounds including equivalents thereof are useful in methods of the present invention for altering an epileptic syndrome by treating an individual in need thereof. Examples of an epileptic syndrome (to be altered or prevented) include epilepsy, Rasmussen's syndrome and West syndrome. Other syndromes which are multi-system disorders but with the primary disability resulting from neurological effects including epilepsy, are considered epileptic syndromes for purposes of the present invention. An example of such a syndrome is tuberous sclerosis syndrome.

As used herein, the term “altering” in the phrase “altering an epileptic syndrome by treatment of an individual” refers to any of a variety of positive effects from the treatment. Such positive effects include, for example, stopping progression of the syndrome, slowing progression of the syndrome, eradicating a complication associated with the syndrome, relieving to some extent a complication associated with the syndrome, and prolonging the survival time of the recipient of the treatment. The treatment may be used in conjunction with one or more therapies for an epileptic syndrome. Use of the treatment in conjunction with another therapy may be to provide two therapies each acting on their own to treat the epileptic syndrome, or may be to provide two therapies where one enhances the effectiveness of the other (e.g., increases the efficacy of the other or improves the outcome from the other) to treat the epileptic syndrome.

A compound is administered in an amount effective for treatment to an individual possessing an epileptic syndrome. Such individuals are mammals capable of possessing an epileptic syndrome. Examples of such mammals are humans, canines, felines and equines. Canines, for example, have typically been treated with phenobarbital or potassium bromide, which can cause severe side effects or be ineffective.

The above described compounds including equivalents thereof are also useful in methods of the present invention for preventing an epileptic syndrome in an individual at risk for developing an epileptic syndrome. Such individuals are mammals at risk for developing an epileptic syndrome. Examples of such mammals are humans, canines, felines and equines. There are a variety of ways by which to identify an individual at risk for developing an epileptic syndrome. For example, prevalence of an epileptic syndrome within a family may be used to identify an individual at risk. Particularly at risk is an identical twin where the other twin has an epileptic syndrome. Another individual at higher risk is a child of biological parents who both have the same epileptic syndrome. In addition to genetic risk factors, there are non-genetic factors that may aid in the identification of an individual at risk for developing an epileptic syndrome. For example, an individual having suffered a closed head injury (e.g., during military service) or a stroke are at increased risk. Another example of an individual at increased risk is a child who experienced an infection resulting in a high internal temperature. Another way, for example, to identify an individual at risk may be changes in the individual's EEG, or the occurrence of a single seizure along with other risk factors. Where an individual is identified to be at risk, one or more of the compounds described herein may be administered as prophylactic therapy in an amount effective to prevent development of an epileptic syndrome. Typically an individual undergoing prophylactic therapy would be monitored periodically for any adverse reactions and any changes suggesting that the therapy was no longer effective.

The above described compounds may be administered in a manner appropriate to the disorder to be treated. Appropriate dosages and a suitable duration and frequency of administration may be determined by such factors as the condition of the patient, the type and severity of the patient's disease and the method of administration. In general, an appropriate dosage and treatment regimen provides the compound(s) in an amount sufficient to provide therapeutic and/or prophylactic benefit. Within particularly preferred embodiments of the invention, a compound may be administered at a dosage ranging from 0.001 to 1000 mg/kg body weight (more typically 0.01 to 1000 mg/kg), on a regimen of single or multiple daily doses. Appropriate dosages may generally be determined using experimental models and/or clinical trials. In general, the use of the minimum dosage that is sufficient to provide effective therapy is preferred. Patients may generally be monitored for therapeutic effectiveness using assays suitable for the condition being treated, which will be familiar to those of ordinary skill in the art.

At least one (i.e., one or more) of the above described compounds may be administered in combination with at least one (i.e., one or more) antiepileptic syndrome agent, e.g., anticonvulsant agent. The compound may function independent of the agent, or may function in coordination with the agent, e.g., by enhancing effectiveness of the agent or vice versa. In addition, the administration may be in conjunction with one or more other therapies for reducing toxicities of therapy. For example, at least one (i.e., one or more) agent to counteract (at least in part) a side effect of therapy (e.g., anticonvulsant therapy) may be administered. Drugs (chemical or biological) that promote recovery or enhancement of appetite, or counteract nausea or fatigue, are examples of such agents. At least one compound described herein may be administered before, after or simultaneous with administration of at least one agent or at least one agent to reduce a side effect of therapy. Where administration is simultaneous, the combination may be administered from a single container or two (or more) separate containers.

The following Examples are offered by way of illustration and not by way of limitation.

### EXAMPLES

### Example 1

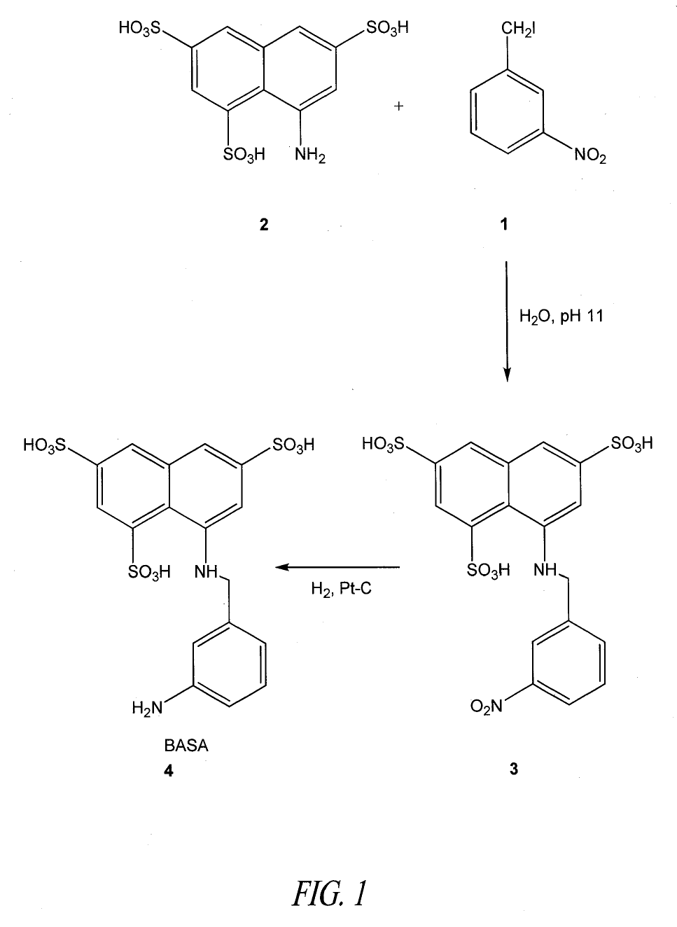

### Synthesis of BASA (FIG. 1)

### Synthesis of Compound 4

3-nitro-benzyl iodide (1) (48.3 g) is added to an aqueous solution (pH 11) of commercially available, 8-aminonaphthalene-1,3,5-trisulfonic acid (2) (29.5 g) with stirring at room temperature (RT). The pH of the solution is adjusted to 1 and after evaporation of the solvent, the product 3 (6.4 g) is precipitated out from ethanol.

Platinum catalyzed hydrogenation of compound 3 affords compound 4 (the benzylamino sulfonic acid or “BASA” of FIG. 1) in 96% yield.

### Example 2

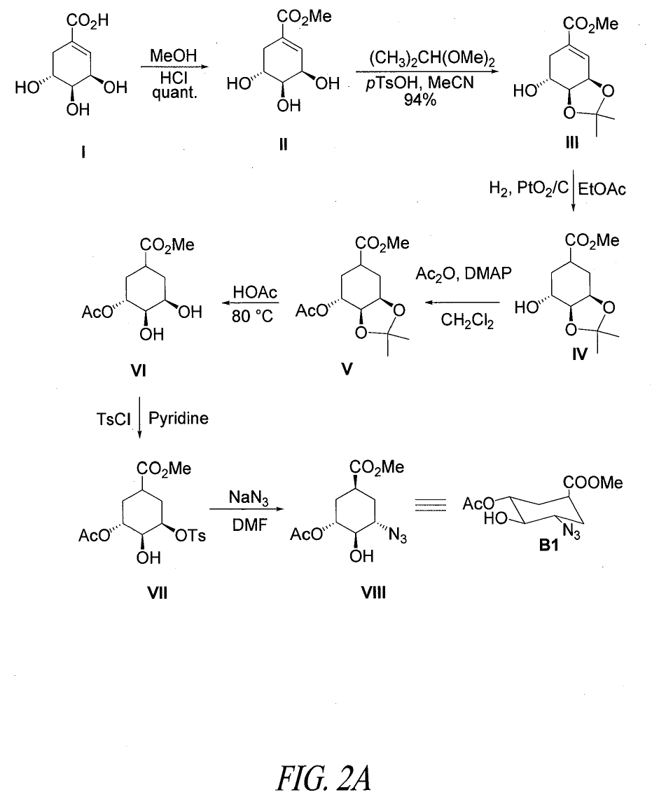

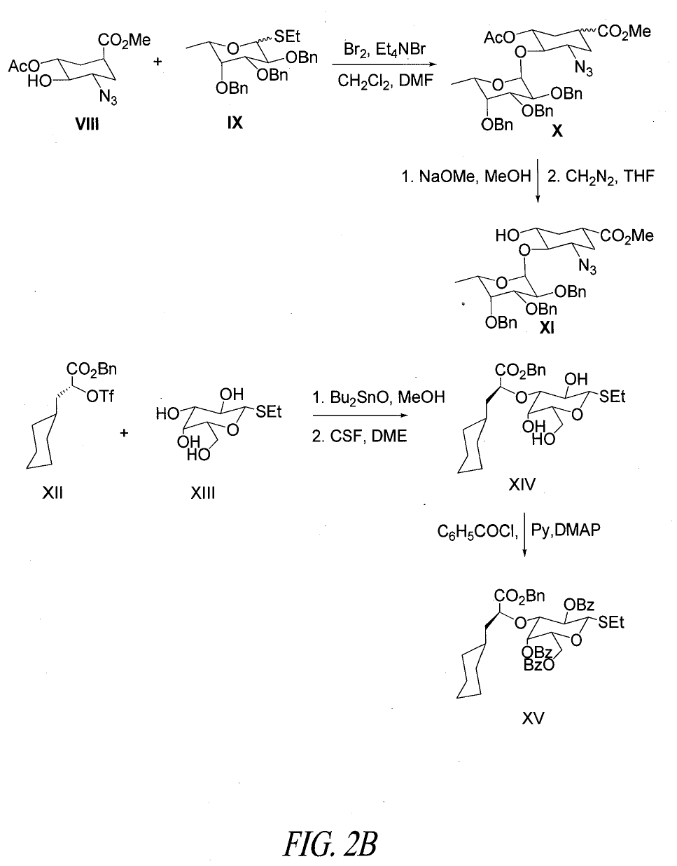

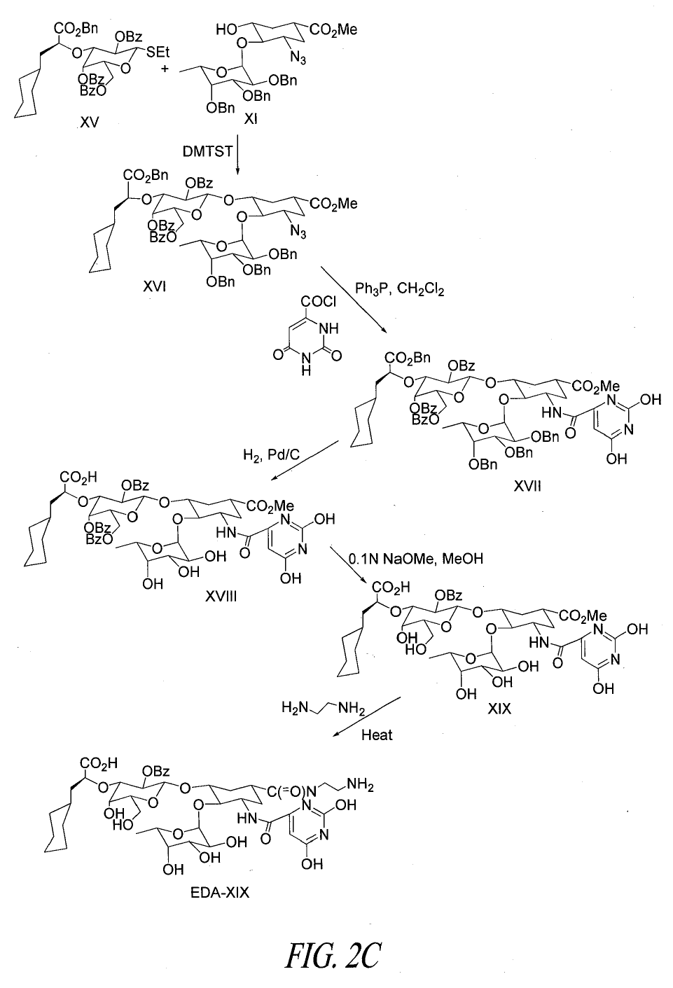

### Synthesis of Glycomimetic (FIG. 2)

### Synthesis of Intermediate II

(−)-Shikimic acid (I, 20 g) in MeOH (200 ml) and sulfuric acid (2 ml, 98%) are stirred at RT for 50 h. The reaction mixture is neutralized with 2N aqueous NaOH in the cold. After evaporation to dryness, the residue is purified by silica gel chromatography to afford II (19.2 g).

### Synthesis of Intermediate III

Methyl shikimate (II, 10 g), 2,2 dimethoxypropane (10 ml) and p-TsOH (0.8 g) are dissolved in acetonitrile (125 ml) and stirred at RT for 1 h. The reaction mixture is then neutralized with triethylamine (2 ml) and evaporated to dryness. The residue is chromatographed on silica gel to yield III (11 g).

### Synthesis of Intermediate IV

The shikimic acid derivative III (10 g) and PtO₂/C (10%, 250 mg) in MeOH (40 ml) are hydrogenated at RT under vigorous stirring. After 16 h the reaction mixture is filtered over celite and evaporated to dryness. The residue is chromatographed on silica gel to yield IV.

### Synthesis of Intermediate V

To a solution of IV (8 g) in DCM (100 ml) at 0° C. are added pyridine (12 ml), acetic anhydride (7 ml) and a DMAP (25 mg). The reaction mixture is stirred at RT for 1 h, and diluted with EtOAc (250 ml). After washing with 0.5 M aqueous HCl (3×50 ml), saturated solution of KHCO₃ (3×50 ml) and brine (3×50 ml), the combined organic layers are dried (Na₂SO₄) and evaporated to dryness. The residue is purified by chromatography on silica gel to yield V (6.8 g).

### Synthesis of Intermediate VI

A solution of V (6.0 g) in acetic acid (30 ml, 80%) is stirred at 80° C. for 1 h. Solvent is evaporated off and the residue is purified by chromatography on silica gel (DCM/MeOH 14:1) to yield VI (3.6 g).

### Synthesis of Intermediate VII

A solution of VI (3 g) and p-TsCl (3.5 g) in pyridine (30 ml) is stirred at RT for 6 h. MeOH (5 ml) is added and the solvent is evaporated at reduced pressure, the residue dissolved in EtOAc (3×150 ml) and the organic layers are washed with 0.5 M aqueous HCl (0° C.), water (cold) and brine (cold). The combined organic layers are dried (Na₂SO₄), filtered on Celite and evaporated to dryness. The residue is purified by chromatography on silica gel (toluene/EtOAc 4:1) to yield VII (3.7 g).

### Synthesis of Compound VIII

A solution of VII (3 g) and NaN₃ (2.5 g) in DMF (20 ml) is stirred at 80° C. The reaction mixture is cooled to RT and diluted with EtOAc (200 ml) and water (50 ml). The organic layer is additionally washed twice with water (2×50 ml) and once with brine (50 ml). All aqueous layers are extracted twice with EtOAc (2×50 ml). The combined organic layers are dried with Na₂SO₄, filtered and the solvent is evaporated off. The residue is purified by chromatography on silica gel (petroleum ether/EtOAc 5:2) to give VIII (2.2 g).

### Synthesis of Compound X

To a solution of ethyl 2,3,4-tri-O-benzyl-α-L-fucothiopyanoside IX (1.5 g) in DCM (3 ml), bromine (150 μl) is added at 0° C. under argon. After 5 min the cooling bath is removed and the reaction mixture is stirred for an additional 25 min at RT. Cyclohexene (200 μl) is added and the reaction mixture is added to a solution of VIII (400 mg), (Et)₄NBr (750 mg) and powdered 4 Å molecular sieves in DCM (10 ml) and DMF (5 ml). After 16 h, triethylamine (1.5 ml) is added and stirred for an additional 10 min, diluted with EtOAc (50 ml) and washed with sat. aqueous NaHCO₃, water and brine. The aqueous layers are extracted twice with EtOAc (2×50 ml). The combined organic layers are dried (Na₂SO₄), filtered and evaporated to dryness. The residue is purified by chromatography on silica gel (toluene/EtOAc 9:1) to yield X (700 mg).

### Synthesis of Compound XI

To a solution of X (1.5 g) in MeOH (20 ml) is added freshly prepared NaOMe (80 mg) and the reaction mixture is stirred in a pressure tube at 80° C. for 20 h. The reaction mixture is cooled to RT and neutralized with acetic acid. Solvent is evaporated to dryness and the residue is dissolved in ether. Freshly prepared diazomethane is added and the excess diazomethane is neutralized with acetic acid. Solvent is evaporated off to give XI (1.25 g).

### Synthesis of Building Block XV

This synthesis is done exactly in same way as described previously (Helvetica Chimica Acta 83:2893-2907 (2000)).

### Synthesis of Compound XVI

A mixture of XI (1.6 g), XV (3 g) and activated powdered molecular sieves 4 Å (1 g) in DCM (17 ml) is stirred at RT under argon for 2 h. Then DMTST (2 g) is added in 4 equal portions over a period of 1.5 h. After 24 h the reaction mixture is filtered over Celite and the filtrate is diluted with DCM (100 ml). The organic layer is washed with sat. aqueous NaHCO₃ and brine and the aqueous layers are extracted twice with DCM. The combined organic layers are dried (Na₂SO₄), filtered and evaporated to dryness. The residue is purified by chromatography on silica gel (toluene/EtOAc 8:1) to yield XVI (1.5 g).

### Synthesis of Compound XVII

To a solution of XVI (500 mg) and orotic acid chloride (500 mg) in dichloromethane (10 ml) is added a solution of triphenylphosphine (500 mg in 5 ml dichloromethane) dropwise during 10 min. The reaction mixture is stirred at RT for 25 h and the solvent is evaporated off. The residue is purified (chromatography on silica gel DCM/MeOH 19:1) to give XVII (250 mg).

### Synthesis of Compound XVIII

To a solution of XVII (200 mg) in dioxane-water (5:1, 12 ml) is added 10% Pd—C (100 mg) and the reaction mixture is stirred vigorously under hydrogen (55 psi) for 24 h. Catalyst is filtered through a bed of celite and the solvent is evaporated off. Residue is purified by silica gel chromatography to give compound XVIII (150 mg).

### Synthesis of XIX

To a solution of compound XVIII (145 mg) in MeOH (5 ml) is added a solution of NaOMe in MeOH (25%, 0.025 ml) and the reaction mixture is stirred at RT for 4 h, neutralized with acetic acid and the solvent is evaporated off. Residue is dissolved in water and passed through a bed of Dowex 50wX-8 (Na-form) resin. Water wash is evaporated off to afford compound XIX (100 mg).

### Synthesis of EDA-XIX

XIX (80 mg) is heated at 70° C. with ethylenediamine (EDA) (1 ml) with stirring for 5 h. Solvent is evaporated off and the purified by sephadex G-25 column to give EDA-XIX (82 mg).

### Example 3

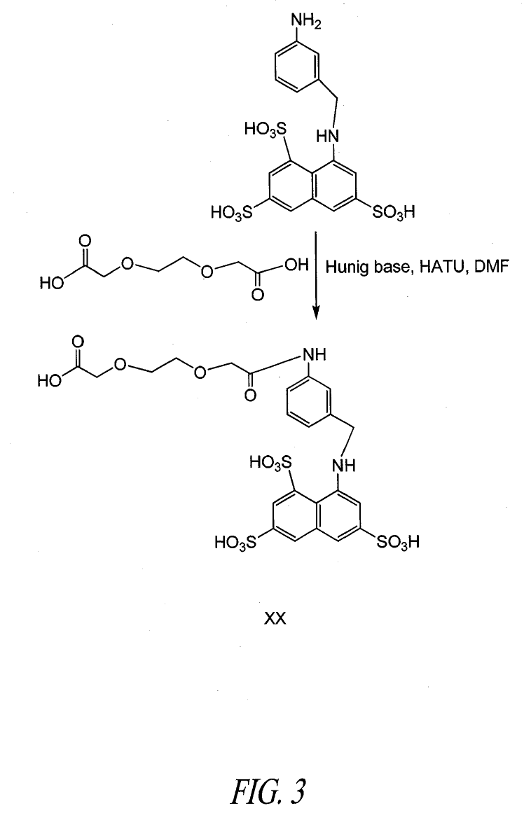

### Synthesis of Pegylated BASA (FIG. 3)

To a solution of 3,6-dioxaoctanedioic acid (PEG, 200 mg, available commercially) in DMF (1 ml) is added Hunig base (0.4 ml), and then HATU (0.35 g) is added after 5 min. The solution is stirred at RT for 10 min. and then a solution of the BASA of Example 1 (50 mg) in DMF (0.1 ml) is added. The reaction mixture is stirred for 4 h at RT and the solvent is evaporated off. The residue is purified by hplc (reverse-phase C18 column) to give XX (40 mg).

### Example 4

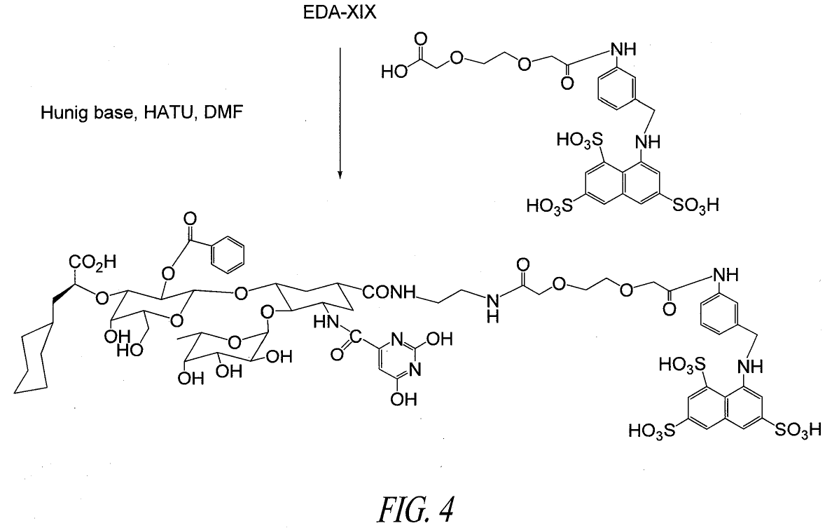

### Synthesis of Glycomimetic-BASA Compound #1 (FIG. 4)

To a solution of XX from Example 3 (0.015 g) in DMF (0.1 ml) is added Hunig base (0.015 ml) and then HATU (0.007 g). The reaction mixture is stirred for 10 min at RT. A solution of EDA-XIX from Example 2 (0.010 g in DMF ml) is added and the reaction mixture is stirred at RT for 8 h. Solvent is evaporated off and the residue is purified by sephadex G-25 chromatography to give the Glycomimetic-BASA #1 of FIG. 4 (0.008 g).

### Example 5

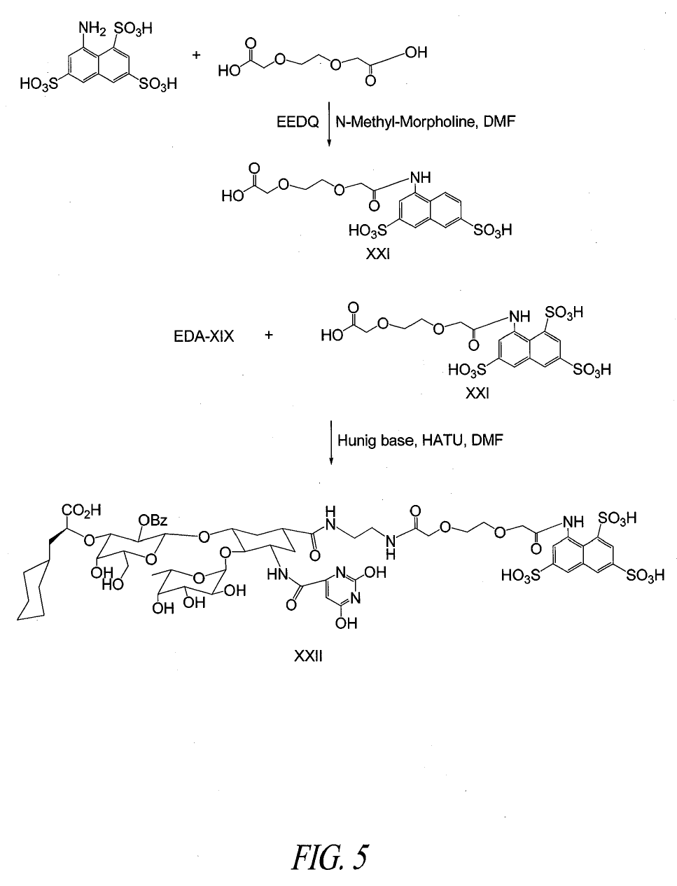

### Synthesis of Glycomimetic-BASA Compound #2 (FIG. 5)

### Synthesis of Compound XXI

To a solution of 3,6-dioxaoctanedioic acid (PEG, 200 mg, available commercially) in DMF (1 ml) is added Hunig base (0.4 ml) and then HATU (0.35 g) is added after 5 min. The solution is stirred at RT for 10 min and then a solution of 8-aminonaphthalene-1,3,6-trisulfonic acid (50 mg, available commercially) in DMF is added. The reaction mixture is stirred for 4 h at RT and the solvent is evaporated off. The residue is purified by hplc (reverse-phase C18 column) to give XXI (25 mg).

### Synthesis of Compound XXII

This synthesis is performed in the same way as described in example 4 except using EDA-XIX from example 2 and XXI to give compound XXII (the Glycomimetic-BASA #2 of FIG. 5) (4 mg).

### Example 6

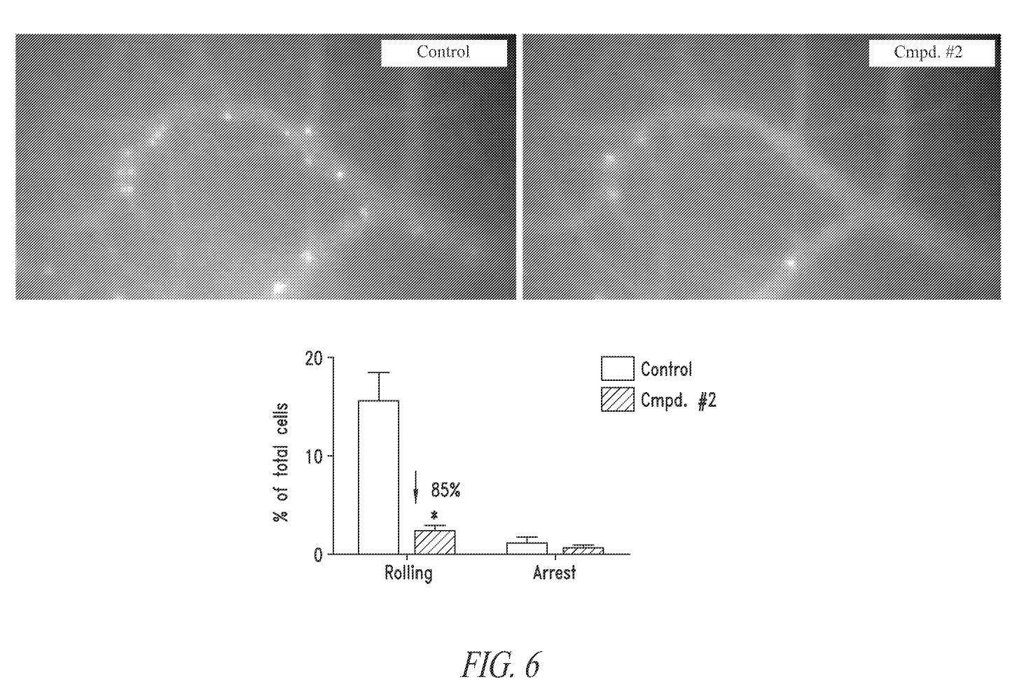

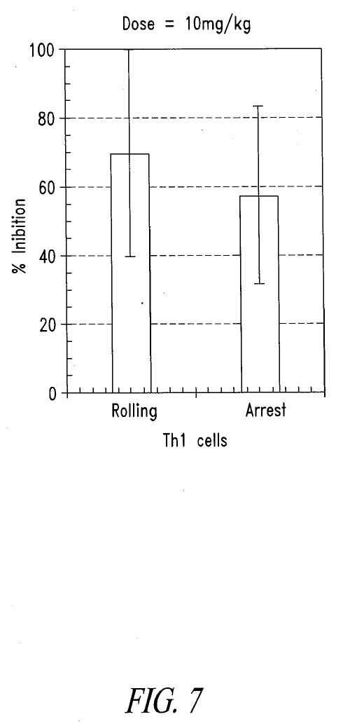

### Effects of Compound #2 on Migration of Neutrophils or T_{H}1 Cells after Status Epilepticus Induction (FIGS. 6 and 7)

Epilepsy is induced by intravenous administration of pilocarpine in mice. After 2 hours of status epilepticus, the mice enter a quiescent phase lasting 4 to 7 days followed by chronic recurring epileptic seizures. Mice treated with Cmpd. #2 directly after experiencing status epilepticus, were examined for effects on the rolling and arrest of neutrophils (FIG. 6) or T_{H}1 cells (FIG. 7) in cerebral vessels at the blood brain barrier by intravital microscopy. Cmpd. #2 inhibited the rolling and arrest of both neutrophils and T_{H}1 cells on the cerebral vessel endothelium.

### Example 7

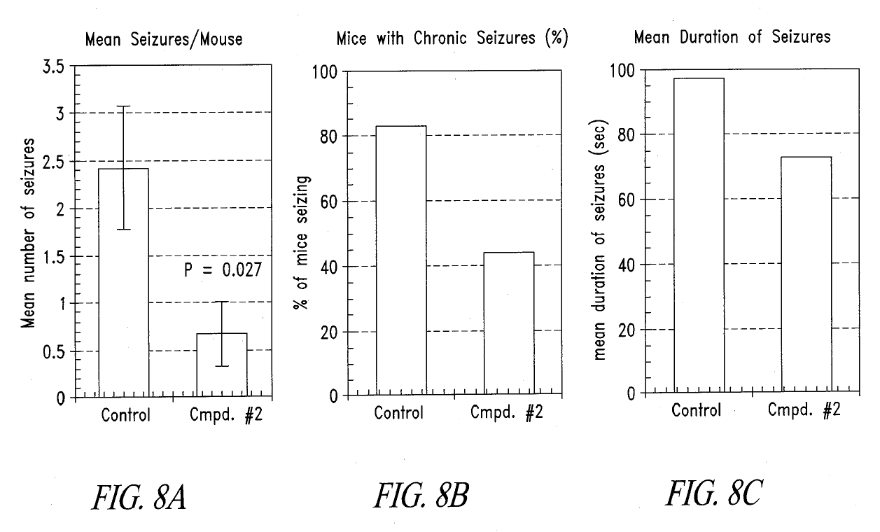

### Effects of Intermittent Dosing of Compound #2 on Treatment of Mice after Status Epilepticus Induction (FIG. 8)

The effects of treatment with Cmpd. #2 after status epilepticus on chronic recurring seizures were also determined by measuring the mean number of seizures/mouse (FIG. 8A), the percentage of mice with chronic seizures (FIG. 8B) and the mean duration of seizures (FIG. 8C). Cmpd. #2 inhibited seizure activity as determined by all three different measures.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

1. A method for altering an epileptic syndrome by treatment of an individual who is in need thereof, comprising administering to the individual a compound in an amount effective for treatment, the compound with the formula: 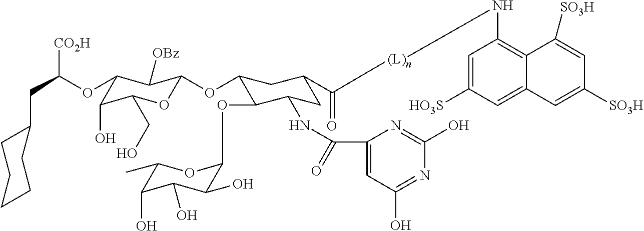
wherein
L=linker group; and
n=0-1.

2. The method according to claim 1 wherein in the compound n=0.

3. The method according to claim 1 wherein in the compound n=1 and L is 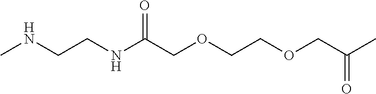 [SMILES: CNCCNC(=O)COCCOCC(C)=O]
where the N of L is attached to terminal C of C(═O) of the compound.

4. The method according to claim 1 wherein in the compound n=1 and L is 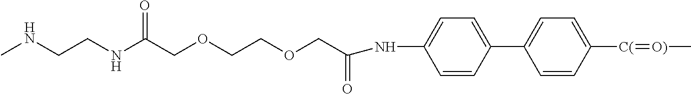
where the N of L is attached to terminal C of C(═O) of the compound.

5. The method according to claim 1 wherein in the compound n=1 and L is 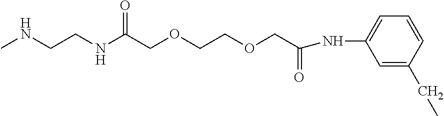 [SMILES: CCc1cccc(NC(=O)COCCOCC(=O)NCCNC)c1]
where the N of L is attached to terminal C of C(═O) of the compound.

6. The method according to claim 1 wherein the individual possesses Rasmussen's syndrome.

7. The method of any one of claims 1-6 wherein the compound is in combination with a pharmaceutically acceptable carrier or diluent.

8. A method for preventing an epileptic syndrome in an individual at risk for developing an epileptic syndrome, comprising administering to the individual a compound in an amount effective for the prevention, the compound with the formula: 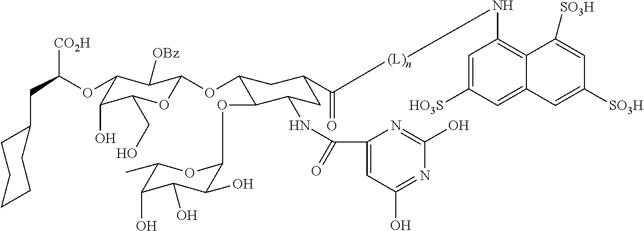
wherein
L=linker group; and
n=0-1.

9. The method according to claim 8 wherein in the compound n=0.

10. The method according to claim 8 wherein in the compound n=1 and L is 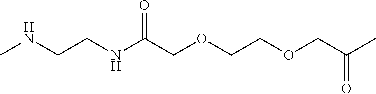 [SMILES: CNCCNC(=O)COCCOCC(C)=O]
where the N of L is attached to terminal C of C(═O) of the compound.

11. The method according to claim 8 wherein in the compound n=1 and L is 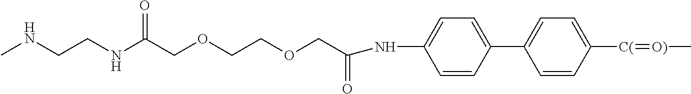
where the N of L is attached to terminal C of C(═O) of the compound.

12. The method according to claim 8 wherein in the compound n=1 and L is 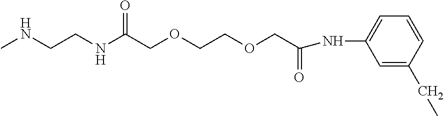 [SMILES: CCc1cccc(NC(=O)COCCOCC(=O)NCCNC)c1]
where the N of L is attached to terminal C of C(═O) of the compound.

13. The method according to claim 8 wherein the individual is at risk for developing Rasmussen's syndrome.

14. The method of any one of claims 8-13 wherein the compound is in combination with a pharmaceutically acceptable carrier or diluent.
